(19) United States
(12) Patent Application Publication
(10) Pub. No.: US 2006/0281724 A1
(43) Pub. Date: Dec. 14, 2006

(54) Methods for Treating Shock

(76) Inventor: Roger M. Loria, Richmond, VA (US)

Correspondence Address: CASTELLANO MALM FERRARIO & BUCK PLLC, 1250 CONNECTICUT AVENUE NW, SUITE 200, WASHINGTON, DC, 20036, US

(21) Appl. No.: 11/422,809

(22) Filed: Jun. 7, 2006

Related U.S. Application Data
(60) Provisional application No. 60/595,126, filed on Jun. 8, 2005.
(60) Provisional application No. 60/702,574, filed on Jul. 26, 2005.

(51) Int. Cl. A61K 31/57

(52) U.S. Cl. 514/178

(57) ABSTRACT

The invention relates to methods of treating shock in a subject by administering a pharmaceutically effective amount of androstenetriol (AET), androstenediol (AED) or derivatives thereof to a subject suffering from or exhibiting the symptoms of shock. In one particular embodiment, the subject is suffering from hemorrhagic shock. The invention also relates to methods of preventing shock in a subject at risk suffering from shock by administering a pharmaceutically effective amount of androstenetriol or a derivative thereof to the subject, prior to or immediately at the onset of the first symptoms of shock. In one particular embodiment, the subject is at risk suffering from hemorrhagic shock.

### CROSS-REFERENCE TO RELATED APPLICATIONS

This application claims priority to U.S. Provisional Application Ser. Nos. 60/595,126, Jun. 8, 2005, and 60/702,574, which are incorporated by reference in their entirety.

### STATEMENT REGARDING FEDERALLY SPONSORED RESEARCH OR DEVELOPMENT

Part of the work performed during development of this invention utilized U.S. Government funds from the Office of Naval Research (Contract No. N00014-03-1-0362). The U.S. Government has certain rights in this invention.

### BACKGROUND OF THE INVENTION

1. Field of the Invention

The invention relates to a method of treating shock in a subject by administering a pharmaceutically effective amount of androstenetriol (AET) androstenediol (AED) or derivatives thereof to a subject suffering from or exhibiting the symptoms of shock.

2. Background of the Invention

Shock, or circulatory insufficiency leading to inadequate blood flow to vital organs, is a potentially life-threatening medical emergency that often leads to organ damage, cardiac arrest, respiratory failure and death.

There are various etiologies of shock, which include cardiogenic shock, hypovolemic shock and vasodilatory shock. These dysfunctions in circulation can in turn be caused by bacterial blood infection (septic shock), severe allergic reaction (anaphylaxis), trauma (traumatic shock), severe bleeding (hemorrhagic shock), or neurologic dysfunction causing abnormal opening of blood vessels (neurogenic shock). While any shock is serious, septic shock and hypovolemic shock are particularly dangerous due to their frequency of occurrence, and due to frequently inadequate treatment regimens. Indeed, despite attempts to improve survival of septic patients with intensive medical care, including antibiotics, aggressive intravenous fluids, nutrition, mechanical ventilation, and surgical interventions, the mortality rate is still quite high.

Trauma or hemorrhagic shock results in activation of the hypothalamic-pituitary-adrenal axis to mediate a cascade of neurohormonal changes as a defensive mechanism. A prolonged state of shock, however, leads to a hypermetabolism, hypoperfusion and immunosuppression, setting the stage for subsequent sepsis, organ damage, multiple organ failure (MOF), cardiac arrest, respiratory failure and death.

The mortality rate in patients with hypovolemic shock is also high, with the cause of death generally being attributed to circulatory collapse due to severe hemorrhage. Traumatic injury and blood loss induce irreversible circulatory shock and represent a major clinical problem, particularly in combat casualties. Traumatic injury (often accompanied by severe blood loss) is the principal cause of death in patients aged 18-44 years and the overall leading cause of life-years lost in the United States. Traumatic injury accounts for millions emergency room situations, millions of hospital admissions, and is estimated to cause 150,000 deaths each year. Although more effective prevention measures will reduce the early deaths resulting from massive hemorrhage and central nervous system injury, the transition from reversible to irreversible hypovolemia, or circulatory collapse, appears to play a major role in the majority of late deaths after trauma and blood loss.

Accordingly, better treatments are needed to increase survival rates of subjects undergoing shock, particularly septic shock and hypovolemic shock. Androstenetriol (AET) and androstenediol (AED), which are physiological metabolites of dehydroepiandrosterone (DHEA), may markedly upregulate the host immune response and prevent immune suppression and modulates inflammation, which can improve survival after bacterial and viral infections, as well as after high doses of radiation. AET, AED and derivatives thereof thus holds promise for reducing side effects and mortality associated with shock.

### SUMMARY OF THE INVENTION

The invention relates to methods of treating shock in a subject by administering a pharmaceutically effective amount of androstenetriol (AET) androstenediol (AED) or derivatives thereof to a subject suffering from or exhibiting the symptoms of shock. In one particular embodiment, the subject is suffering from hemorrhagic shock.

The invention also relates to methods of preventing shock in a subject at risk suffering from shock by administering a pharmaceutically effective amount of androstenetriol or a derivative thereof to the subject, prior to or immediately at the onset of the first symptoms of shock. In one particular embodiment, the subject is at risk suffering from hemorrhagic shock.

### BRIEF DESCRIPTION OF THE DRAWINGS

FIG. 1 depicts the reduction in mortality rates in response to administration of AET in two hemorrhagic rat models. In the “blood loss model,” mortality in AET-treated animals was reduced to zero percent three days after experiencing hemorrhagic conditions that reduced total blood volume by 40%. In the “pressure reduction model,” mortality in the AET-treated animals was reduced from 75% in control animals to 43% two days after experiencing hemorrhagic conditions that lowered mean arterial pressure to about 35-40 mmHg (˜60% reduction).

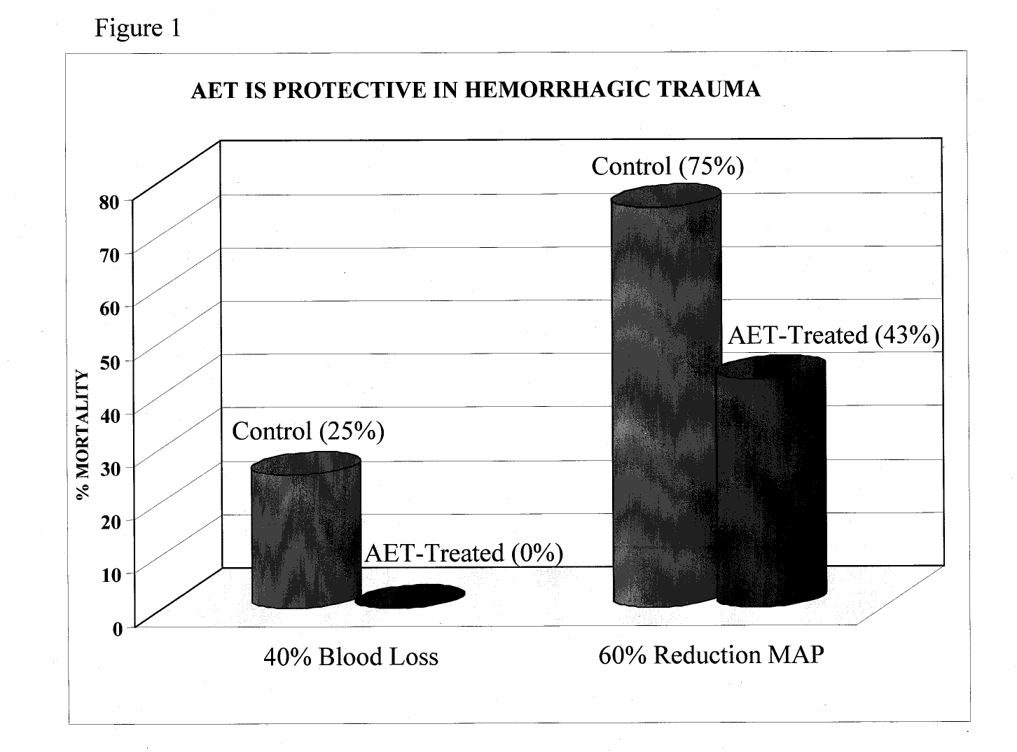

### DETAILED DESCRIPTION OF THE INVENTION

The invention relates to methods of treating shock in a subject by administering a pharmaceutically effective amount of androstenetriol (AET) androstenediol (AED) a derivatives thereof to a subject suffering from or exhibiting the symptoms of shock. As used herein, the term “treatment” is used to indicate a procedure which is designed ameliorate one or more causes, symptoms, or untoward effects of an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. Thus, a treatment may include treating a subject to attenuate symptoms such as, but not limited to, discomfort, pain, tachycardia, bradycardia, oliguria, confusion and other mental disturbances, thirst and death in a subject, or may include removing or decreasing the severity of the root cause of the abnormal condition in the subject. Treatment of shock also includes treating after-arising symptoms that are related to the initiation shock, such as reperfusion injury. The injury arising from interrupted blood flow is a two component system—tissue injury occurring during the ischemic interval, and injury occurring during reperfusion that follows ischemia. When there is a long duration of ischemia, the “direct” damage resulting from hypoxia alone is the predominant mechanism. For shorter duration's of ischemia, the indirect or reperfusion mediated damage becomes increasingly more important. As used herein, the term “subject” is used interchangeably with the term “patient,” and is used to mean an animal, in particular a mammal, and even more particularly a non-human or human primate.

Specifically, methods of the current invention are directed towards the treatment of shock in a subject. Shock is used herein to describe the general medical condition in which organs and/or tissues of the body of the subject are not receiving an adequate flow of blood. Although the progression from the initial onset of shock to the ultimate endpoint of shock—death—is a gradual process, there are currently three recognized stages of shock. In one embodiment of the present invention, the methods are used to treat the initial stage, Stage I shock, Stage II shock or Stage III shock. In one particular embodiment, the methods of the present invention are used to treat the initial stage of shock. The initial stage of shock is characterized by cardiac output insufficient to meet the body's metabolic needs, but not otherwise low enough to produce significant symptoms. The patient may be anxious and alert, with increased respiration. In another particular embodiment, the methods of the present invention are used to treat Stage I shock. Stage I of shock (“compensated shock” or “non-progressive shock”) occurs when the body detects decreased blood flow or perfusion and begins to activate several reactive mechanisms to restore perfusion or direct blood flow to the most vital body organs. Stage I shock can also be asymptomatic, but may also include, but is not limited to such symptoms as low blood flow or perfusion, rapid or increased heart rate, shallow or irregular breathing, hypotension, hypertension, pallor and cyanosis. In another particular embodiment, the methods of the present invention are used to treat Stage II shock. Stage II of shock (“decompensated shock” or “progressive shock”) occurs when the compensatory mechanisms of the body begin to fail and organ perfusion can not be restored to normal or maintained. Symptoms of Stage II shock include, but are not limited to, confusion, anxiety, disorientation and other mental disturbances indicating a lack of oxygen to the brain, chest pains, increased heart rate, oliguria, multiple organ dysfunction, falling blood pressure, rapid breathing, weakness and pupil dilation. In still another particular embodiment, the methods of the present invention are used to treat Stage III shock. Stage III of shock (“irreversible shock”) occurs after the state of decreased perfusion or blood flow has existed to such an extent that the organs and tissues of the body are permanently affected. Such symptoms include, but are not limited to, multiple organ failure, kidney failure, coma, blood pooling in the extremities and death.

In additional embodiments of the current invention, the methods are used to treat cardiogenic shock, hypovolemic shock and vasodilatory shock, each of which can be in any of the three aforementioned stages of shock. In one particular embodiment of the present invention, the methods are used to treat cardiogenic shock. Cardiogenic shock is, generally speaking, low blood flow or perfusion that is caused by heart malfunction where the heart does not pump adequate blood. Causes can include any condition that interferes with ventricular filling or emptying, such as, but not limited to, embolism, ischemia, regurgitation and valve malfunction. In another particular embodiment of the present invention, the methods are used to treat vasodilatory shock. Vasodilatory shock is caused by severe venous or arteriolar dilation, which results in inadequate blood flow. Several known causes contribute to vasodilatory shock including, but not limited to, cerebral trauma, drug or poison toxicity, anaphylaxis, liver failure, bacteremia and sepsis. In another more particular embodiment of the present invention, the methods are used to treat shock resulting from sepsis or bacteremia. In an even more particular embodiment, the methods are used to treat septic shock or bacteremic shock in either stage I, II or III. In yet another embodiment, the methods of the present invention are used to treat hypovolemic shock. Hypovolemic shock is, generally speaking, decreased intravascular volume; and the decrease in intravascular volume can be relative or absolute. Hemorrhage from conditions such as, but not limited to, ulcers, trauma, accidents, surgery, and aneurysm causes hypovolemic shock; but loss of other body fluids may also cause hypovolemic shock. For instance, renal fluid loss, intravascular fluid loss, water or other peritoneal fluid loss may contribute to hypovolemic shock. In one particular embodiment of the present invention, the methods are used to treat hemorrhagic shock. In an even more particular embodiment, the methods are used to treat hemorrhagic shock in either stage I, II or III.

The invention provides methods of treating or preventing hemorrhagic shock in a patient, which includes administering to a patient diagnosed as suffering from blood loss. The blood loss may, but not need, be measured as a percentage of the subject's blood volume, such as, for example, a blood loss of greater than about 15% total blood volume, or greater than 20%, 25%, 30%, 35%, 40%, or 50% of the subject's total volume. In other terms, the blood loss may, but not need, be measured in terms of a drop in blood volume in any amount sufficient to cause hemorrhagic shock in a particular subject, such as, for example, a loss of about 1000 ml, of about 1500 ml, or of about 2000 ml or more in a human subject. The blood loss may also be measured in terms of a drop in systolic blood pressure, such as, for example, a drop in systolic blood pressure that is about 20 mmHg, 30 mmHg, 40 mmHg, 50 mmHg, 60 mmHg, 70 mmHg, 80 mmHg, 90 mmHg or 100 mmHg or more than 100 mmHg lower than the subject's normal systolic blood pressure. In particular embodiments, the subject is undergoing or has undergone a medical procedure, such as, but not limited to, surgery, a transfusion or child birth.

The treatment methods may optionally include monitoring the subject for symptoms of hemorrhagic shock both before and after administration of AET, AED or derivatives thereof.

Methods of treating or preventing shock described herein comprise administering a pharmaceutically effective amount of AET, AED or derivatives thereof to a subject. As used herein, the term “administer” and “administering” are used to mean introducing at least one compound into a subject. When administration is for the purpose of treatment, the substance is provided at, or after the onset of, a symptom of shock. The therapeutic administration of this substance serves to attenuate any symptom, or prevent additional symptoms from arising. When administration is for the purposes of preventing shock (“prophylactic administration”), the substance is provided in advance of any visible or detectable symptom. The prophylactic administration of the substance serves to attenuate subsequently arising symptoms or prevent symptoms from arising altogether. The route of administration of the compound includes, but is not limited to, topical, transdermal, intranasal, vaginal, rectal, oral, subcutaneous intravenous, intraarterial, intramuscular, intraosseous, intraperitoneal, epidural and intrathecal.

Furthermore, the methods of treating or preventing shock of the present invention also relate to coadministering one or more substances in addition to the AET ,AED or derivatives thereof to the subject. The term “coadminister” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. The scope of the invention is not limited by the identity of the substance which may be coadministered. For example, AET or AED may be coadministered with an AET or AED derivative or other pharmaceutically active substances, such as catecholamines or other a, adrenergic agonists, α₂ adrenergic agonists, β adrenergic agonists or β₂ adrenergic agonists, including but not limited to epinephrine, norepinephrine, dopamine, isoproterenol, vasopressin and dobutamine. Alternatively, AET, AED or derivatives thereof, may be coadministered with fluids or other substances that are capable of alleviating, attenuating, preventing or removing symptoms in a subject suffering from, exhibiting the symptoms of, or at risk of suffering from hypovolemic shock, vasodilatory shock or cardiogenic shock, such as, but not limited to, trans-sodium crocetinate (TSC). Types of fluid that can be coadministered with AET, AED or derivatives thereof should be specific to the circumstances surrounding the particular subject that is suffering from, exhibiting the symptoms of, or at risk of suffering from shock. For example, fluids that may be coadministered with AET or AED include but are not limited to, salt solutions —such as sodium chloride and sodium bicarbonate—as well as whole blood, plasma, serum, serum albumin and colloid solutions. Colloid solutions include, but are not limited to, solutions containing hetastarch, albumin or plasma. In one particular embodiment of the present invention, fluids selected from the group of salt solutions, colloidal solutions, whole blood, plasma and serum are coadministered with AET, AED or derivatives thereof in patients suffering from or exhibiting the symptoms of a hypovolemic shock, such as hemorrhagic shock.

Particular embodiments of the coadministration methods of the present invention include methods of performing a transfusion in a subject, with the transfusion methods comprising providing blood that comprises AET, AED or derivatives thereof to a subject. The blood used in the transfusion methods can be whole blood or any fractionated portion, e.g. plasma, serum, red blood cells, etc., thereof.

Methods of the present invention relate to administering a pharmaceutically effective amount of AET, AED or derivatives thereof to a subject. As used herein, the term “pharmaceutically effective amount” is used to mean an amount of a substance that can elicit a desired response without excessive side effects. The response to the pharmaceutically effective amount may be a cellular, organ or tissue-specific response, or system response.

The methods of the present invention relate to administering AET, AED or derivatives thereof. As used herein, “androstenetriol” is used to mean trihydroxy-androst-5ene. One example of AET includes, but is not limited to 3β, 7β, 17β-trihydroxy-androst-5ene or 3β, 7α, 17β-trihydroxy-androst-5ene. Other examples of AET include but are not limited to 3β, 16α, 17-trihydroxy-androst-5ene, 3β, 15β, 17β-trihydroxy-androst-5ene, 2α, 3α, 17β-trihydroxy-androst-5ene and 3β, 6α, 17β-trihydroxy-androst-5ene. As should be apparent, the term “AET” also encompasses the α isomers and the β isomers, or racemic mixtures thereof. Stereochemistry of AET is based upon a comparison with cholest-5-ene-3β, 7β-diol and cholest-5-ene-3β, 7β-diol.

In addition to AET, the invention encompasses the use of derivatives of AET to treat or prevent shock in a subject. Derivatives of AET, and their synthesis, are described in U.S. Pat. No. 5,641,768, which is hereby incorporated by reference. Derivatives of AET also include metabolic intermediates thereof, and AET may also be substituted with protective groups which, on hydrolysis, yield AET. Hence, acylated and alkylated derivatives are useful as precursors to AET. Compounds such as those of the formula (1): 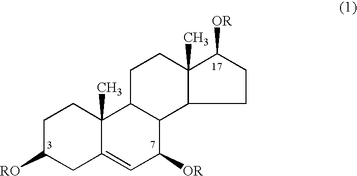
wherein R may be H, alkenyl of 2-8 carbons, alkyl of 1-8 carbons, phenylalkyl of 1-4 carbons, phenyl or COR₂, wherein R₂ is H; alkyl of 1-8 carbons, alkenyl of 2-8 carbons, phenylalkyl wherein the alkyl has 1-4 carbons (including benzyl) or phenyl. Any phenyl moiety may have up to three substituents chosen from among hydroxy, carboxy of 1-4 carbons, halo, alkoxy of 1-4 carbons, alkyl of 1-4 carbons, or alkenyl of 2-4 carbons and wherein any alkyl may be a straight chain, branched chain, or the alkyl may be wholly or partially cyclized.

The instant invention also relates to the use of 5-androstene (AED), which can exist in at least two epimeric forms: 5-androstene-3β-17α-diol (αAED) and 5-androstene-3β-17β-diol (βAED). Similar to AET, the term “AED” encompasses both the α isomers and the β isomers, or racemic mixtures thereof. AED, as well as its esters and ethers, inhibit growth and accelerate cell aging, induce apoptosis and death of gliomas, and in particular glioblastomas. The structures of the α and β isomers of AED are given below in formulas 2 and 3, respectively: 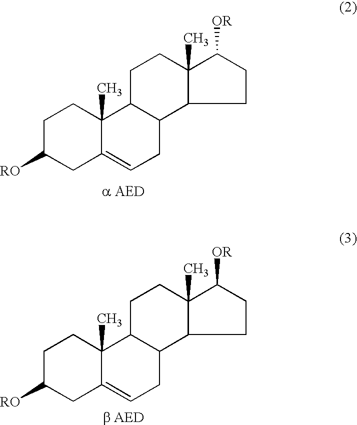
wherein R is a hydrogen. The invention also encompasses derivatives of AED wherein R may be H, alkenyl of 2-8 carbons, alkyl of 1-8 carbons, phenylalkyl of 1-4 carbons, phenyl or COR₂, wherein R₂ is H; alkyl of 1-8 carbons, alkenyl of 2-8 carbons, phenylalkyl wherein the alkyl has 1-4 carbons (including benzyl) or phenyl. Any phenyl moiety may have up to three substituents chosen from among hydroxy, carboxy of 1-4 carbons, halo, alkoxy of 1-4 carbons, alkyl of 1-4 carbons, or alkenyl of 2-4 carbons and wherein any alkyl may be a straight chain, branched chain, or the alkyl may be wholly or partially cyclized. The R groups need not be identical.

The methods of the present invention also relate to administering dehydroepiandrosterone (DHEA) or derivatives thereof to treat or prevent shock. Derivatives of DHEA also include metabolic intermediates thereof.

Other derivatives of AET or AED, which may be used in the methods of the present invention include, but are not limited to those compounds having the structure of formula 5, 6, 7, 8, 9, 10, 11, 12, 13 or 14 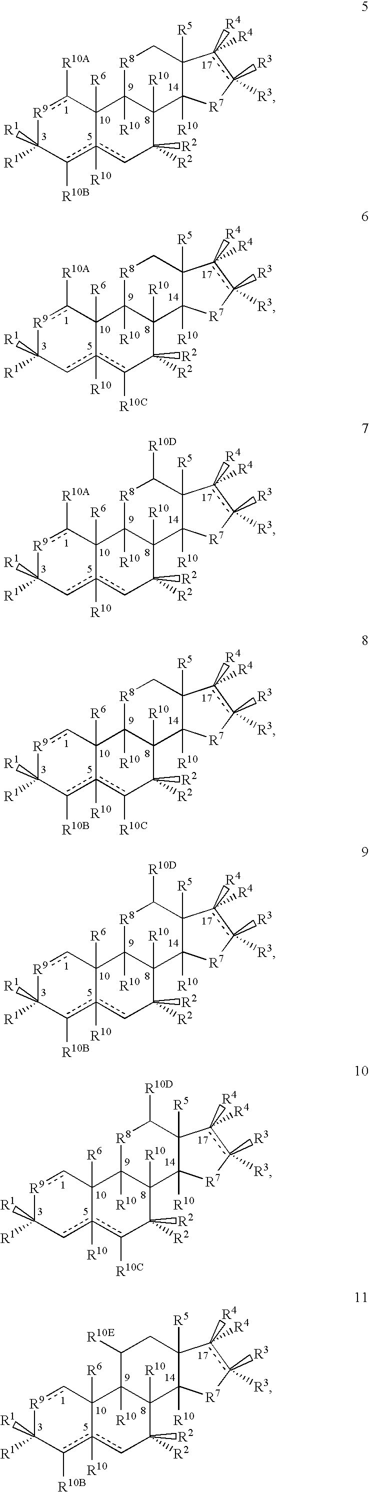 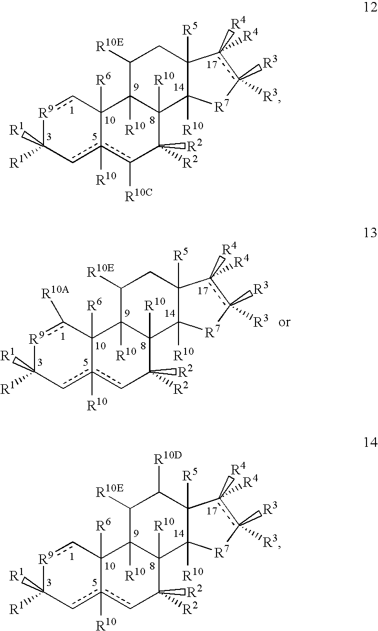
or a metabolic precursor or a metabolite thereof, wherein

R¹⁰ moitieties at the 5 (if present), 8, 9 and 14 positions prespectively are in the α,α,α,α; α,α,α,β; .α,α,β,α; α,β,α,α,β,α,α,α; α,α, β,β; .β,α,β,α; α,β,α, β; β,α,α,β; β,β,α,α; α,β,β,α; α,β,β,β; β,α,β,β; β,β,α,β; β,β,β,α; β,β,β,β configurations.

wherein R^{10A}, R^{10B}, R^{10C}, R^{10D} R^{10E} respectively are in the α,α;α,β;β,α or β, β configurations,

wherein, each R¹, R², R³, R⁴, R⁵, R⁶, R¹⁰, R^{10A}, R^{10B}, R^{10C}, R^{10D} and R^{10E} independently are —H, —OH, —OR^{PR}, SR^{PR}, —N(R^{PR})₂, —O—Si—(R¹³)₃, —CHO, —CHS, —CN, —SCN, —NO₂, —NH₂, —COOH, —OSO₃H, —OPO₃H, an ester, a thioester, a thionoester, a phosphoester, a phosphothioester, a phosphonoester, a phosphiniester, a sulfite ester, a sulfate ester, an amide, an amino acid, a peptide, an ether, a thioether, an acyl group, a thioacyl group, a carbonate, a carbamate, a halogen, an optionally substituted alkyl group, an optionally substituted alkenyl group, an optionally substituted alkynyl group, an optionally substituted aryl moiety, an optionally substituted heteroaryl moiety, an optionally substituted heterocycle, an optionally substituted monosaccharide, an optionally substituted oligosaccharide, a nucleoside, a nucleotide, an oligonucleotide, a polymer, or,

one more of R¹, R², R³, R⁴, R⁵, R⁶, R¹⁰, R^{10A}, R^{10B}, R^{10C}, R^{10D} and R^{10E} are ═O, ═S, ═N—OH, OH, ═CH₂, ═CH—CH₃, or an independently selected spiro ring and the hydrogen atom or the second variable group that is bonded to the same carbon atom is absent, or,

one or more of two adjacent R¹-R⁶, R¹⁰, R^{10A}, R^{10B}, R^{10C}, R^{10D} and R^{10E} comprise an independently selected epoxide, acetal, a thioacetal, ketal or thioketal;

R⁷ is —C(R¹¹)₂—, —C(R¹¹)₂—C(R¹¹)₂—, —C(R¹¹)₂—C(R¹⁰)₂—C(R¹¹)₂—, —C(R¹¹)₂—O—C(R¹⁰)2—, —C(R¹⁰)₂—S—C(R¹⁰)2—, —C(R¹⁰)₂—NR^{PR}—C(R¹⁰)₂—, —O—, —O—C(R¹⁰)₂—, —S—, —S—C(R¹⁰)₂—, NR^{PR} or —NR^{PR}—C(R¹⁰)₂—;

R⁸ and R⁹ independently are —C(R¹⁰)₂—, —C(R¹⁰)₂—C(R¹⁰)₂—, —O—, —O—C(R¹¹)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring;

R¹³ independently is C₁₋₆ alkyl; and

R^{PR} independently is —H or a protecting group. In typical embodiments, one or two of R^{10A}, R^{10B}, R^{10C}, R^{10D} and R^{10E} are not hydrogen or one R⁴ is —NH₂, an optionally substituted amine, —N(R^{PR})², ═NOH, ═NO-optionally substituted alkyl, an amide or an N-linked amino acid.

Other embodiments include (1) certain new formula 1 compounds, which are new chemical entities, (2) compositions that comprise a formula 1 compound and another compound or an excipient, (3) formulations that comprise a formula 1 compound and 1, 2, 3, 4, 5, 6 or more excipients. The formulations can be designed for human pharmaceutical use or they can be suitable for veterinary use. Therapeutic use embodiments include (1) use of a formula 1 compound for the preparation of a medicament and (2) use of a formula 1 compound for the preparation of a medicament for the prophylaxis or treatment of a condition or symptom disclosed herein. Specific compounds for use in the methods of the present invention include, but are not limited to those compounds listed in U.S. Ser. No. 10/728,400, filed Dec. 5, 2003, which is a continuation-in-part of U.S. Ser. No. 10/651,515, filed Aug. 28, 2003, both of which are incorporated by reference in their entirety.

The invention also relates to methods of preventing or preventing the progression of shock in a subject at risk suffering from shock by administering a pharmaceutically effective amount of AET, AED or a derivative thereof to the subject, prior to or immediately at the onset of the first symptoms of shock. As used herein, the term “prevent,” as it relates to shock, indicates that a substance of the present invention is administered to a subject to prohibit one or more symptoms of shock from detectably appearing or to attenuate the effects of one or more symptoms of shock. Of course, the term “prevent” also encompasses prohibiting entirely shock or any of its associated symptoms, from detectably appearing. Thus a subject may be “pretreated,” such as a subject in a surgical setting, by using the substances of the present invention to prevent shock from arising. The phrase “preventing the progression,” as it relates to shock, is used to mean a procedure designed to prohibit the detectable appearance of one or more additional symptoms of shock in a patient already exhibiting one or more symptoms of shock, and is also used to mean prohibiting the already-present symptoms of shock from worsening in the subject. The symptoms of shock that are included in preventative methods of the present invention include, but are not limited to, such symptoms of shock as highlighted herein, such as tachycardia, shallow or erratic breathing and death. A subject that is “at risk of shock” may be recognized based upon the specific circumstances surrounding a subject. For example, a surgery patient or a subject that has been wounded and begun losing blood would be “at risk of shock.” Similarly, a patient with a bacterial infection also exhibiting a fever or low blood pressure, may also be “at risk of shock.”

In additional embodiments of the current invention, the methods are used to prevent cardiogenic shock, hypovolemic shock and vasodilatory shock, each of which can be in any of the three aforementioned stages of shock. In one particular embodiment of the present invention, the methods are used to prevent cardiogenic shock. In another particular embodiment of the present invention, the methods are used to prevent vasodilatory shock. In another more particular embodiment of the present invention, the methods are used to prevent shock resulting from sepsis or bacteremia. In an even more particular embodiment, the methods are used to prevent septic shock or bacteremic shock in either stage I, II or III. In yet another embodiment, the methods of the present invention are used to prevent hypovolemic shock. In one particular embodiment of the present invention, the methods are used prevent hemorrhagic shock. In an even more particular embodiment, the methods are used to prevent hemorrhagic shock in either stage I, II or III.

Similar to the methods of treating shock described herein, one embodiment of the methods of preventing shock of the present invention comprises coadministering another substance with AET, AED or a derivative thereof. The scope of the invention is not limited by the identity of the substance which may be coadministered with AET or AED to prevent shock. For example, AET or AED may be coadministered with an AET or AED derivative or other pharmaceutically active substances, such as catecholamines or other α₁ adrenergic agonists, α₂ adrenergic agonists, β adrenergic agonists or β₂ adrenergic agonists, including but not limited to epinephrine, norepinephrine, dopamine, isoproterenol, vasopressin and dobutamine to prevent shock. Alternatively, AET, AED or derivatives thereof, may be coadministered with fluids or other substances that are capable of preventing or removing symptoms in a subject at risk of suffering from hypovolemic shock, vasodilatory shock or cardiogenic shock. The types of fluid that can be coadministered with AET, AED or derivatives thereof to prevent shock should be specific to the circumstances surrounding the particular subject that is at risk of suffering from shock. For example, fluids that may be coadministered with AET or AED include but are not limited to, salt solutions—such as sodium chloride and sodium bicarbonate—as well as whole blood, plasma, serum, serum albumin and colloid solutions. Colloid solutions include, but are not limited to, solutions containing hetastarch, albumin or plasma. In one particular embodiment of the present invention, fluids selected from the group of salt solutions, colloidal solutions, whole blood, plasma and serum are coadministered with AET or a derivative thereof in subjects at risk of suffering a hypovolemic shock, such as hemorrhagic shock.

In another aspect, the invention includes whole blood, or a fractionated component thereof, comprising AET, AED or derivatives thereof. For example, the invention includes whole blood or a blood component in a vessel (such as a blood bag suitable for a transfusion procedure), wherein the whole blood or blood component comprises AET, AED or derivatives thereof. The vessel may optionally include a label indicating that the whole blood or blood component can be used to treat or prevent shock, such as, but not limited to hemorrhagic shock. Similarly, the invention also provides methods of preparing whole blood or a component thereof, with the preparation methods comprising adding an effective amount of AET, AED or a derivative thereof to treat or prevent shock in a subject that is suffering from or at risk of suffering from shock.

A medicament comprising a substance of the present invention, for example, AET, AED or a derivative thereof, may be prepared by standard pharmaceutical techniques known in the art, depending upon the mode of administration and the particular disease to be treated. The medicament will usually be supplied as part of a sterile, pharmaceutical composition which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. Specific examples of excipients include but are not limited to poly-ethylene glycol (PEG), dimethyl sulfoxide (DMSO), ethanol and mixtures thereof. For the preparation of suspensions oils (e.g. vegetable oils) may be used to provide oil-in-water or water in oil suspensions. In certain situations, delayed release preparations may be advantageous and compositions which can deliver, for example, AET, AED or a derivative thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by ionophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colorants, odorants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the disease or disorder to be treated, the age and condition of the individual to be treated, etc. and a physician will ultimately determine appropriate dosages to be used.

The following examples are meant to be illustrative and not intended to limit the scope of the invention described herein.

### EXAMPLE 1

### Rat Model of Volume Hemorrhagic Trauma

Twenty-four rats were subjected to 40% loss of total blood volume, consisting of catheterization and laparotomy (soft tissue injury) to mimic trauma and hemorrhage. One hour after onset of hemorrhage, the animals were resuscitated with crystalloid fluid and packed red blood cells (PRBCs). Twelve animals received one subcutaneous injection of AET in a methyl cellulose suspension at a concentration of 40 mg/kg body weight in 100 μl/kg body weight, one hour after initiation of hemorrhage, but prior to fluid resuscitation. Twelve animals received subcutaneous methyl cellulose control injection at 100 μl/kg body weight. Three days after induction of hemorrhage, the twelve animals that received AET had a 100% survival rate; whereas the mortality rate was 25%, in the untreated group (P<0.04, Barnard's unconditional test of superiority using difference of two binomial proportions).

### EXAMPLE 2

### Rate Model of Blood Pressure Hemorrhagic Trauma

In a second model of hemorrhagic trauma, 15 rats were hemorrhaged as in Example 1 down to a mean arterial pressure of about 35-40 mmHg and resuscitated one hour from onset of hemorrhage with crystalloid and PRBCs. Seven animals received one animals received one subcutaneous injection of AET in a methyl cellulose suspension at a concentration of 40 mg/kg body weight in 100 μl/kg body weight, one hour after initiation of hemorrhage, but prior to fluid resuscitation. Eight animals received subcutaneous methyl cellulose control injection at 100 μl/kg body weight. Two days after induction of hemorrhage, mortality in the untreated group (n=8) was 75%. The mortality rate in the AET-treated animals was 43%, demonstrating that AET was still protective in cases of hemorrhagic trauma where system pressure is severely reduced.

Statistical analysis shows no differences in the response to AET between the two studies in Example 1 and Example 2, respectively.

What is claimed is:

1. A method of treating symptoms in a subject suffering from shock, said method comprising administering a pharmaceutically effective amount of adrostenetriol (AET), androstenediol (AED) or a derivative thereof to said subject.

2. The method of claim 2, wherein the AET is administered.

3. The method of claim 2, wherein said shock is selected from the group consisting of hypovolemic shock, vasodilatory shock and cardiogenic shock.

4. The method of claim 3, wherein said shock is hypovolemic shock.

5. The method of claim 4, wherein said hypovolemic shock is hemorrhagic shock.

6. The method of claims 5, wherein the route of administration is selected from the group consisting of oral, intravenous, interperitoneal, and subcutaneous.

7. The method of claim 6, wherein the AET is administered with poly-ethylene glycol (PEG).

8. A method of preventing shock in a subject at risk of said shock, said method comprising administering s pharmaceutically effective amount of adrostenetriol (AET), androstenediol (AED) or a derivative thereof to said subject.

9. The method of claim 8, wherein the AET is administered.

10. The method of claim 9, wherein said shock is selected from the group consisting of hypovolemic shock, vasodilatory shock and cardiogenic shock.

11. The method of claim 10, wherein said shock is hypovolemic shock.

12. The method of claim 11, wherein said hypovolemic shock is hemorrhagic shock.

13. The method of claims 12, wherein the route of administration is selected from the group consisting of oral, intravenous, interperitoneal, and subcutaneous.
